(19) United States
(12) Patent Application Publication
(10) Pub. No.: US 2026/0000605 A1
(43) Pub. Date: Jan. 1, 2026

(54) CONTACT LENSES WITH POROUS ANNULUS FOR DELIVERING BIOLOGICS

(71) Applicant: Colorado School of Mines, Golden, CO (US)

(72) Inventors: Anuj Chauhan, Golden, CO (US); Zachary Sparks, Golden, CO (US)

(21) Appl. No.: 19/225,804

(22) Filed: Jun. 2, 2025

Related U.S. Application Data
(60) Provisional application No. 63/654,671, filed on May 31, 2024.

(51) Int. Cl. A61K 9/00; A61K 9/70; A61K 47/32

(52) U.S. Cl. CPC A61K 9/0051; A61K 9/7007; A61K 47/32

(57) ABSTRACT

Methods of loading biologics onto contact lenses for use as drug-delivering ophthalmic devices are provided, as are contact lenses made by such methods and methods of treatment employing such contact lenses, while maintaining optical clarity and structural rigidity. The lens material has at least one concentric layer consisting of a porous material with pores large enough to deliver a biologic to a subject over a desired time period of diffusion. The concentric layers are formed through polymerizing a first, outer layer in a rotating cylinder and then repeating the method the desired number of times with different formulations for the different layers.

### CROSS-REFERENCE TO RELATED APPLICATION

This application claims the benefit of priority under 35 U.S.C. § 119 (e) to U.S. provisional patent application 63/654,671, filed 31 May 2024, the entirety of which is incorporated herein by reference.

### STATEMENT REGARDING FEDERALLY SPONSORED RESEARCH

This invention was made with partial government support under grant number R01EY034477 awarded by the National Institute of Health of the United States Department of Health and Human Services. The government has certain rights in the invention.

### FIELD

This disclosure relates generally to intraocular drug delivery, and particularly to ophthalmic devices, such as contact lenses, and methods of use and manufacturing thereof that enable controlled, extended, and/or sustained release of a drug into an eye of a subject.

### BACKGROUND

Anterior segment ocular diseases are commonly treated by instillation of eye drops despite many deficiencies including rapid clearance from the ocular surface within a few minutes which leads to a low bioavailability. Many researchers have developed contact lenses for sustained release of drugs and shown an improvement in delivery compared to drops due to an increase in residence time. Commercial contact lenses alone are usually not suitable for sustained release due to the high drug diffusivity. Thus, several technologies have been developed to attenuate drug release, such as incorporation of nanoparticles or vitamin E nanobarriers, imprinting, and multi-layer lenses. These technologies can provide sustained release of both hydrophobic and hydrophilic small molecule drugs. Many of these technologies achieve sustained release without compromising optical clarity which would allow patients to use the lenses without significant impact on their normal activities.

Delivery of biological therapeutics to the eye has become an area of greater focus as investigations of protein and peptide therapeutics have shown to have higher levels of efficacy in treating ocular conditions compared to small molecule drugs. Biologic therapeutics, such as anti-VEGF drugs, are commonly used to slow the progression of age-related macular degeneration and other conditions impacting the back of the eye. While anti-VEGF is commonly used for managing back of the eye diseases, it could also be useful in treating front of the eye diseases, such as cornea neovascularization. Some biologics have been FDA-approved for treating front of eye diseases such as OXERVATE, which is the first FDA-approved treatment for neurotrophic keratitis. Oxervate is a 0.002% (20 mcg/mL) formulation of Cenegermin, which is a recombinant human nerve growth factor (rhNGF). Cenegermin is a large protein molecule with a molecular weight of 13,266. The typical prescription of OXERVATE involves instillation of 6 eyedrops daily at 2-hour intervals, for eight weeks.

However, current commercial lenses and those designed for sustained release of small molecule drugs are not suitable for delivering biologics because the small pore size in the lenses prevent uptake and release of the large biologics into the lenses. The small pore sizes are a necessary requirement because larger pores of the size of biologics will scatter visible light making the lenses opaque.

Thus, a novel approach is needed to create a contact lens that retains transparency in the vision axis, while having the porosity to load and release biologics. The present disclosure uses a poly hydroxyethyl methacrylate (pHEMA) hydrogel lens with a clear center and a concentric ring of larger pores that allow the uptake and release of proteins without compromising the optical clarity of the lens in the vision zone.

### SUMMARY

In an aspect of the present disclosure, a method for making an ophthalmic device suitable for drug delivery comprises polymerizing a first monomer formulation in a rotating cylinder to form a first and outermost concentric layer of a contact lens.

In some embodiments, the first monomer formulation comprises hydroxyethylmethacrylate (HEMA).

In some embodiments, the method further comprises polymerizing a second monomer formulation in a rotating cylinder to form a second concentric layer.

In some embodiments, at least one of the first and second monomer formulations comprises HEMA.

In some embodiments, the method further comprises polymerizing a third monomer formulation in a rotating cylinder to form a third layer.

In some embodiments, at least one of the first, second, and third monomer formulations comprises hydroxyethylmethacrylate.

In some embodiments, the method further comprises loading a drug into the contact lens.

In some embodiments, the drug is a biologic.

In some embodiments, the biologic is selected from the group consisting of proteins, micro-RNAs, small molecules, and combinations thereof.

In some embodiments, the biologic has a molecular weight from about 1 kDA to about 180 kDA.

In another aspect of the present disclosure, a contact lens suitable for ophthalmic delivery of a drug comprises hydroxyethylmethacrylate and a biologic drug.

In some embodiments, the contact lens comprises three layers.

In another aspect of the present disclosure, a method for administering a biologic drug to a subject in need thereof comprises applying a contact lens as disclosed herein to an eye of the subject, wherein the biologic drug is a protein, a micro-RNA, or a combination thereof, the applying step is carried out for at least a duration of release of the drug from the contact lens, and the duration of release is at least about 2 hours.

In another aspect of the present disclosure, a contact lens comprises a first layer of a non-porous material; and a second layer of a porous material, wherein the first and second layers are concentric.

In some embodiments, the contact lens further comprises a non-porous, optically clear central portion.

In some embodiments, at least one of the following is true: a diameter of the central portion is no more than about 8 mm; and a visible light transmittance of the central portion is at least about 90%.

In some embodiments, the contact lens further comprises a pharmaceutical drug, loaded within the second layer; and a third layer of a non-porous material, overlying the first and second layers on a front or outermost surface of the contact lens and configured to promote one-directional diffusion of the pharmaceutical drug into an eye of a wearer of the contact lens.

In some embodiments, the contact lens further comprises magnetic nanoparticles at one or more predetermined locations within the contact lens.

In some embodiments, the contact lens has a visible light transmittance of at least about 90%.

In another aspect of the present disclosure, a method for administering a pharmaceutical drug to a subject in need thereof comprises applying a contact lens as disclosed herein to an eye of the subject, wherein the pharmaceutical drug is loaded within the second layer of the contact lens, the applying step is carried out for at least a duration of release of the drug from the contact lens, and the duration of release is at least about 2 hours.

In an aspect of the present disclosure, a method for making an ophthalmic device suitable for drug delivery comprises polymerizing a first monomer formulation in a rotating cylinder to form a first layer of a contact lens.

In embodiments, the method may further comprise polymerizing a second monomer formulation in a rotating cylinder to form a second layer. The method may, but need not, further comprise polymerizing a third monomer formulation in a rotating cylinder to form a third layer.

In embodiments, at least one of the first, second, and third monomer formulations may comprise hydroxyethylmethacrylate.

In embodiments, the method may further comprise loading a drug into the contact lens. The drug may, but need not, be a biologic, which may, but need not, be selected from the group consisting of proteins, micro-RNAs, small molecules, and combinations thereof. The biologic may, but need not, have a molecular weight from about 1 kDA to about 180 kDA.

In another aspect of the present disclosure, a contact lens suitable for ophthalmic delivery of a drug comprises hydroxyethylmethacrylate and a biologic.

In embodiments, the contact lens may comprise three layers.

In another aspect of the present disclosure, a method for administering a biologic drug to a subject in need thereof comprises applying a contact lens to an eye of the subject, wherein the contact lens comprises the biologic drug, the biologic drug is a protein, a micro-RNA, or a combination thereof, the applying step is carried out for at least a duration of release of the drug from the contact lens, and the duration of release is at least about 2 hours.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are diagrams illustrating the process for polymerizing single layer hydrogel tubes and the core-annular cylindrical rods with a porous ring.

FIG. 2A shows a top view of single layer polymerized pHEMA hydrogel made with 80% water in precursor. FIG. 2B shows the top view and side view of the hydrogel tube removed from the vial. FIG. 2C shows the hydrogel tube cut open and arch punched for testing the sample.

FIGS. 3A and 3E show the polymerization steps in fabrication of two-layer pHEMA hydrogel tube and concentric layer porous pHEMA.

FIG. 3A shows a clear non-porous pHEMA at the vial bottom.

FIG. 3B shows the first (outer) layer of non-porous concentric pHEMA. FIG. 3C shows a second layer of porous concentric pHEMA.

FIG. 3D shows a two-layer pHEMA tube removed from the vial, cut along cylindrical axis and arch-punched.

FIG. 3E shows a two-layer pHEMA tube filled with non-porous pHEMA to make the concentric porous layer pHEMA rod with a clear center, with the rod removed and then cut into discs.

FIG. 4A shows the radial transport direction for single and two-layer hydrogels as a schematic and image of the sample.

FIG. 4B shows the axial transport direction for concentric layer hydrogel and concentric layer hydrogel polymerized on nonporous layer as a schematic and image of the sample.

FIG. 5 shows potential pore alignments with the different planes and directions referenced throughout the present disclosure.

FIG. 6 shows BSA and γ-globulin releases from multi-concentric layer samples to measure axial transport of the hydrogel formulations.

FIG. 7 shows BSA and gamma globulin releases from single layer pHEMA samples to measure radial transport of the hydrogel formulations.

FIG. 8 shows the partition coefficient, K, and diffusivity, D, of pHEMA hydrogels made from formulation varying in water content from 55 to 80% water.

FIG. 9A shows a comparison of the measured EWC and percent water in formulation.

FIG. 9B shows the dependency of the water volume in pores (%) and the volume of water in the formulation.

FIG. 10A shows the drying dynamics for single layer hydrogels of 1.5-1.6 mm thickness and 19.05 mm diameter.

FIG. 10B shows the drying dynamics for multi-concentric layer hydrogels of 2.5 and 5 mm length.

FIG. 11 shows scanning electron microscopy images of pHEMA hydrogel cross sections polymerized from HEMA formulations of varying water content.

FIG. 12 shows HEMA hydrogels prepared with a range of water, showing the precursors in vials and the resulting hydrogels of that formulation.

FIG. 13 shows the percent transmittance of light through pHEMA hydrogels polymerized as planar sheets in 250 μm thick molds.

FIG. 14A is an image of lathe-cut pHEMA lenses with concentric porous annulus.

FIG. 14B is an image of the lathe-cut pHEMA lenses 3 and 4 held in forceps.

FIG. 15 shows transmittance measurements for the central zone of the lathe-cut lenses.

FIG. 16 are graphs illustrating the release profiles of bovine serum albumin (BSA), Y-globulin, and gold nanoparticles (Au NP) with model fit for the five lens formulations (solid lines), as described in Examples 8 through 10.

FIG. 17 shows the five lathe-cut pHEMA lenses with porous annulus imaged after soaking in a gold nanoparticle solution (1 mg/ml) for visual evidence of the uptake of the particles.

FIG. 18 shows fluorescein release from PLGA microparticles.

FIG. 19A shows PLGA microparticles before release of fluorescein, a model dye.

FIG. 19B shows PLGA microparticles after 65 days of release of fluorescein.

FIG. 20A shows uniformly distributed magnetic microspheres throughout a silicone lens.

FIG. 20B shows uniformly distributed magnetic microspheres throughout a silicone lens after a preliminary test of moving the magnetic microspheres into a ring shape pattern.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

As used herein, unless otherwise specified, the terms “administering” and “administration” include administration methods in which a drug is directly administered to a subject, e.g., by putting the drug directly into a dosage form that is administered to the subject, as well as methods in which the drug is indirectly administered to a subject, e.g., by putting a precursor or prodrug of the drug directly into a dosage form that is administered to the subject.

As used herein, unless otherwise specified, the term “ophthalmic device” refers to a device or a composition of matter that is adapted or configured to be placed in physical contact with the cornea. “Ophthalmic devices,” as that term is used herein, may be provided in the form of one or more contact lenses.

As used herein, unless otherwise specified, the term “pharmaceutical composition” refers to a composition of matter comprising at least one active pharmaceutical ingredient that is adapted or configured to be administered to an animal for a therapeutic purpose.

As used herein, unless otherwise specified, the term “subject” refers generally to an animal, including but not limited to a human, to which a composition, device, or formulation provided by the present disclosure is administered or is to be administered. Other animals that may be “subjects” as those terms are used herein include but are not limited to companion animals, such as cats, dogs, and horses; livestock animals, such as cattle, goats, sheep, and pigs; mice; and rats.

Unless otherwise specified, all references herein to any drug encompass, in addition to the base drug, any and all pharmaceutically acceptable salts, polymers, esters, and acids thereof.

Embodiments of the present disclosure generally include an ophthalmic device capable of sustained release of a biologic. Embodiments of the present disclosure also include methods of administration of such devices to a subject, which in some embodiments is a human, to treat a disease or condition and/or achieve a physiological objective. The compositions and methods exhibit advantageous efficiency and health benefits as compared to prior art compositions, devices, and methods. The compositions, devices, and methods are generally provided to treat, prevent, or reduce the risk of a disease or disorder, including but not necessarily limited to an ophthalmic disease or disorder.

Pharmaceutical compositions provided by the present disclosure may be formulated in a unit dosage form. A unit dosage form refers to a physically discrete unit suitable as a unitary dose for subjects undergoing treatment, with each unit containing a predetermined quantity of the active compound calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a subject at a single point in time or during a time interval.

A dose may be administered in a single dosage form or in multiple dosage forms. When multiple dosage forms are used the amount of compound contained within each dosage form may be the same or different. The amount of active compound contained in a dose may depend on the route of administration and whether the disease in a subject is effectively treated by acute, chronic, or a combination of acute and chronic administration.

The methods of the present disclosure allow for contact lenses capable of loading and releasing biologics at therapeutic rates. A significant advantage of the present disclosure is the ability of the contact lens to be loaded with biologics while maintaining comparable strength, optical clarity, and transmittance to that of commercial contact lenses. As a result, compositions and methods of the present disclosure may be effective to treat or prevent a disease or condition, and/or achieve a physiological objective, without the need for injections and/or in fewer doses than those needed to achieve the same results with prior art compositions, devices, and methods. Use of the methods and systems of the present disclosure may also provide further advantages and benefits, such as less inconvenience and less frequent administration.

Ophthalmic devices of the present disclosure may be provided in any suitable form and physical manifestation. By way of non-limiting example, the ophthalmic devices can be administered to a subject in the form of a contact lens. Ophthalmic devices of the present disclosure may thus comprise any suitable pharmaceutically acceptable additives, binders, and/or fillers, and may also comprise therapeutic agents.

In some embodiments of the present disclosure, ophthalmic devices may be, or may be adapted to be, placed or held on the cornea of a subject for a predetermined length of time before being removed. Such embodiments may particularly include those embodiments in which the ophthalmic device is provided in the form of a contact lens. By way of non-limiting example, the length of time may be from about 1 hour to about 30 days, or alternatively in any range having a lower bound of any whole number of hours from about 1 hour to about 720 hours and an upper bound of any whole number of hours from about 1 hour to about 720 hours. In some embodiments, the length of time may be about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, bout 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days, or any length in any range bounded by any two of these values.

The present inventors have found that administration of a drug to a subject in need thereof by the compositions, devices, and methods of the present disclosure may treat a disease or condition and/or achieve a physiological objective in the subject more effectively than the compositions, devices, and methods of the prior art. Specifically, the cost, inconvenience, and risk of side effects to the subject may be reduced.

Preferred dosages and treatment lengths for the methods of the present disclosure may vary according to the particular disease or condition to be treated and/or the particular physiological objective to be achieved. By way of non-limiting example, administration of compositions and devices according to the present disclosure may be continued for an indefinite period of time, e.g. as a maintenance regimen, for the treatment or prevention of a chronic condition, or for continual or continuous maintenance of one or more tissues or organs, including but not necessarily limited to the eye generally and/or the cornea specifically, in a functional and/or healthy condition. By way of further non-limiting example, administration of compositions and devices according to the present disclosure may be discontinued upon resolving an acute condition or upon achieving a physiological objective of treatment.

The present inventors have also investigated the release profile of two protein biologics (bovine serum albumin (BSA) and γ-globulin) from protein-loaded contact lenses made according to the present disclosure. Gold nanoparticle (NP) release profiles were also measured by the present inventors to demonstrate feasibility of releasing particles from the lenses. The porous gels exhibited diffusion-controlled release of both model proteins. The present inventors therefore conclude that the devices and methods of the present disclosure have the unexpected advantage of having pores large enough to be capable of loading and releasing biologics while maintaining transparency within the field of vision. Methods for manufacturing a contact lens according to the present disclosure include polymerizing a monomer formulation, as discussed below, in a partially filled rotating cylinder to form a ring. The process can be repeated multiple times to manufacture a rod with multiple concentric rings in which at least one ring is porous. In embodiments, the contact lens contains three or more layers. In some embodiments, the contact lens can contain at least one non-porous layer. In some embodiments, the outermost layer is prepared with a 40% water formulation to form a transparent outermost ring. In some embodiments, the second layer is prepared with a 55% water HEMA formulation to form a porous ring. In some embodiments, the third layer is prepared with a water-free monomer formulation.

The concentric porous lenses can, in some embodiments, have a non-porous layer at the front of the lens to direct therapeutic release entirely to the post-lens tear film thereby increasing the efficiency of the delivery of the therapeutic.

Methods for manufacturing a contact lens according to the present disclosure further include a step of loading the lens with a biologic. Additionally, in some embodiments, coloading of polymers with the biologics can further increase the release duration. In some embodiments, small molecules can be released from the center while biologics are released from the porous ring. In some embodiments, the partition coefficient of the biologics in the porous region is increased by integrating monomers with higher affinity for the biologics of interest.

The lenses are immersed in a protein solution at 2 mg/mL or 10 mg/mL for a period of time. The period is generally selected to achieve equilibrium loading of the contact lens, which in embodiments may be between about 10 days and about 18 days and in particular embodiments may be about 14 days.

Contact lenses according to the present disclosure may be “daily wear” (DW) contact lenses adapted to be worn for one waking day and removed before sleeping; “extended wear” (EW) contact lenses adapted to be worn continuously during both waking and sleeping hours for up to six consecutive full days; or “continuous wear” (CW) contact lenses adapted to be worn continuously during both waking and sleeping hours for up to 30 consecutive full days. The duration of release of a selected drug from the contact lens may be less than, about equal to, or more than the intended wear cycle of the contact lens.

As a non-limiting example, a contact lens according to the present disclosure can comprise an outermost (first) layer of non-porous material to provide structural rigidity, a second porous layer, a third layer of non-porous material for improved layer adhesion, and a central portion. In embodiments, this central portion can be no more than about 8 mm in diameter. In embodiments, the second porous layer is where biologics are loaded. In embodiments, the degree of porosity can be adjusted by changing the water content in the formulation. A larger water content increases the volume of pores but that can also impact the structural integrity of the lens. The radius of the central zone should be sufficiently large to minimize the possibility of interference with vision. The pupil size in humans varies from 2-4 mm in diameter in bright light but it increases to about 4-8 mm in the dark, and so the central zone can be about 8-mm in diameter to maximize visible range.

### Hydrogels

Hydrogels are soft polymeric networks with significant water content that leads to biologically relevant properties, which are useful in several biomedical applications including tissue engineering, contact lenses, drug delivery, sensors etc. In most of these applications, the pore size of the gels plays an important role in impacting physical properties such as water content, modulus, and diffusion rates of molecules.

In some embodiments, the hydrogel comprises hydroxyethylmethacrylate (HEMA). HEMA can be polymerized to form poly (2-hydroxyethyl methacrylate)(pHEMA). pHEMA is a biocompatible, optically transparent, hydrophilic, and non-degradable polymer.

In embodiments, the monomer formulation further comprises a photoinitiator and/or a crosslinker.

### Water Content and Pore Size

The pore size of the hydrogel plays an important role in impacting physical properties such as water content, modulus, and diffusion rates of molecules. Delivery of larger biologics (e.g., proteins and micro-RNA) and cell growth for tissue engineering requires hydrogels with pore size ranging from hundreds of nanometers to microns. The monomer mixture can also include water which acts as the solvent for the polymerization mixture. The amount of water in the formulation is important because a full hydrated pHEMA hydrogel has a water content of about 40% (w/w) based on the wet basis, which means that the weight of water is 40% of the total wet weight. If the monomer formulation includes more than 40% water, the excess water will phase separate during polymerization, and form pores dispersed throughout the matrix. Thus, the porosity of the pHEMA hydrogels can be controlled by adjusting the amount of water in the monomer formulation. A hydrogel prepared with a monomer formulation that has less water than the saturation content will not have any large pores because the entirety of the water is used for hydrating the polymerized gel. A non-porous HEMA hydrogel has pores approximately 2-5 nm in size.

Layers of contact lenses and similar ophthalmic devices according to the present disclosure, and/or made by methods according to the present disclosure, may include water content in the monomer formulation in any amount from about 0.0 wt % to about 60 wt %, or alternatively in any range having a lower bound of any whole number of tenths of a weight percent from 0.0 wt % to 60 wt % and an upper bound of any whole number of tenths of a weight percent from 0.0 wt % to 60 wt %. In some embodiments, the water content of the monomer formulation used to make a layer of a contact lens according to the present disclosure may be about 0 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 14.5 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, about 29 wt %, about 30 wt %, about 31 wt %, about 32 wt %, about 33 wt %, about 34 wt %, about 35 wt %, about 36 wt %, about 37 wt %, about 38 wt %, about 39 wt %, about 40 wt %, about 41 wt %, about 42 wt %, about 43 wt %, about 44 wt %, about 45 wt %, about 46 wt %, about 47 wt %, about 48 wt %, about 49 wt %, about 50 wt %, about 51 wt %, about 52 wt %, about 53 wt %, about 54 wt %, about 55 wt %, about 56 wt %, about 57 wt %, about 58 wt %, about 59 wt %, or about 60 wt %, or any amount in any range bounded by any two of these values. As described throughout this disclosure, the water content of a layer may be controlled, optimized, selected, and/or tuned to provide for a desired pore size and thus provide the desired release profile and/or duration of release of a selected drug from the contact lens.

### Polymerization

In order to maintain optical clarity of the lens in the vision zone, it is advantageous to polymerize a pHEMA lens with a clear center and a concentric ring of larger pores that allows the uptake and release of proteins.

In some embodiments, the lenses comprise at least two layers. In some embodiments, the lenses comprise at least three layers. In some embodiments, the lenses include an outermost layer of non-porous pHEMA to provide structural rigidity. In some embodiments, the lenses include a layer that covers all the concentric layers to provide one-directional diffusion.

In embodiments, stepwise polymerization is performed in a rotating mold to manufacture cylindrical rods with a clear central zone and a porous annulus. Polymerization is repeatable in order to form distinct layers. In embodiments, polymerization occurs via UV light exposure. In some embodiments, polymerization can occur through any method known in the art.

### Lens Formation

In order to form contact lenses, the hydrogel rod is cut into buttons of approximately 6 mm length. The buttons are soaked in water to allow for HEMA monomer exchange in which the unpolymerized HEMA monomer in the hydrogels diffuses out. The buttons are dried and lathe cut into contact lenses. In some embodiments, the fully hydrated lenses can be dried at room temperature. The lathe-cut lenses were again soaked in water in order to extract any unpolymerized monomer. The lenses were then characterized by measuring water content and transmittance.

Additionally, 3D printing may be used for manufacturing lenses, as it may be possible to 3D-print a lens with a porous annulus.

### Biologics

The contact lenses of the present disclosure may be used to load and release a large variety of biologics. In some embodiments, the biologics may comprise micro-RNAs. In some embodiments, the biologics may comprise peptides. In some embodiments, the biologics may comprise proteins. In embodiments, the biologics have a molecular weight greater than 1 kilodalton (kDA). In embodiments, the biologics may have a molecular weight from about 1 kDA to about 180 kDA, or any size in any range having a lower bound of any whole number of daltons from 1,000 to 180,000 and an upper bound of any other whole number of daltons from 1,000 to 180,000.

In addition, the contact lenses may also be co-loaded with small molecule drugs. The formation of distinct layers within the contact lenses of the present disclosure facilitate delivery of biologics from the annulus and small molecules drugs from the transparent non-porous center.

### Partition Coefficient and Diffusivity

Calculations for the partition coefficient, K, and the diffusivity, D, are described in Sparks, Z. et al., “Polymerization of hydroxyethyl methacrylate (HEMA) under rotation to form core-annular hydrogels,” Journal of Colloid and Interface Science (2025), and in Sparks, Z., et al., “Sustained release of proteins from contact lenses with porous annulus,” Drug Deliv. and Transl. Res. (2025), both of which are incorporated by reference herein in their entirety. These two calculations can be used to characterize the different lens formulations and to infer pore connectivity in desired directions. The partition coefficient is the ratio of the concentration of the protein in the hydrogel to the concentration of the protein in solution at equilibrium.

The disclosure is further described by reference to the following non-limiting examples.

### Examples

### Example 1: Hydrogel Polymerization and Layered Hydrogels

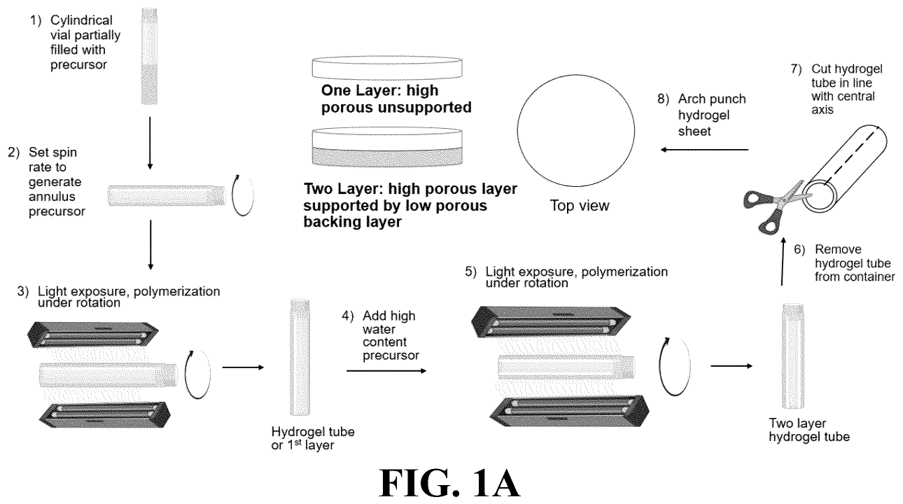

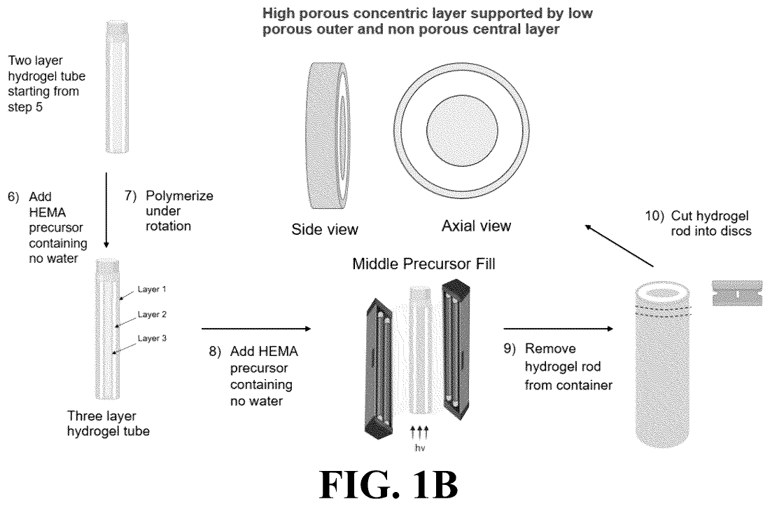

The process for polymerizing single layer hydrogel tubes and the core-annular cylindrical rods with a porous ring is illustrated in FIG. 1.

The monomer 2-hydroxymethacrylate (HEMA), crosslinker ethylene glycol dimethacrylate (EGDMA), and photoinitiator Darocur Diphenyl (2,4,6-trimethylbenzoyl)-phosphine oxide (TPO) were used (Sigma Aldrich). The proteins bovine serum albumin (BSA) and human gamma globulin (γ-Glob), and Dulbecco's phosphate buffered saline (PBS) were used (Sigma Aldrich). Pierce Micro BCA assay reagents were used (Fisher Scientific).

The outermost ring (steps 1-4, FIG. 1A) can be porous or nonporous. Polymerization of the second ring is shown in steps 5-6 of FIG. 1A. The annular tube was cut along the length and punched to obtain discs for transport measurements (FIG. 1A, steps 7-8).

As seen in FIG. 1B, another embodiment of a process for polymerizing single-layer hydrogel tubes and core-annular cylindrical rods with a porous ring can continue from step 5 illustrated in FIG. 1A and includes polymerizing a low porous pHEMA layer inside of the high porous layer followed by filling the hydrogel tube to obtain a multi-concentric layer pHEMA hydrogel rod (FIG. 1B, steps 6-9). Once the core of the hydrogel rod is fully hydrated, the rod may be cut into discs (FIG. 1B, step 10).

A glass or polypropylene vials with inside diameter of approximately 1.9 cm (¾″) were coated on the inside with a hydrophobic layer prior to precursor addition to prevent sticking of polymerized hydrogel to the inside of the vial. A wide range of structures can be prepared with the number of rings and properties of each ring controlled by the number of steps and monomer formulation used in each step. In each step, the vial was partially filled with the chosen monomer formulation with total volume calculated based on the desired thickness of the ring. The partially filled glass vial was placed horizontally in the lab-made tube spinning assembly, as described in Sparks, Z. et al., “Polymerization of hydroxyethyl methacrylate (HEMA) under rotation to form core-annular hydrogels,” Journal of Colloid and Interface Science (2025), which is incorporated by reference herein in its entirety.

The spinning RPM was controlled by the current provided to the 18 VDC brushed motor (Digikey) by a power supply (Eventek, 30V, 5A) which was set to achieve a minimum rotational speed of about 800 rpm and a maximum rotational speed of about 1700 rpm. A light box with mounted CFL UV bulbs was placed over the tube spinning assembly to provide radiation for initiating the polymerization reaction, The duration of time for polymerization was set at 10 minutes for the outermost layer and additional 20 minutes for each extra layer to account for the absorption of light by the polymerized layers. In many of the structures explored here, three concentric layers were polymerized, prior to filling the remaining volume with water-free HEMA monomer solution and polymerizing the rod-core vertically.

### Example 2: Manufacturing Samples for Transport and Water Content Measurements

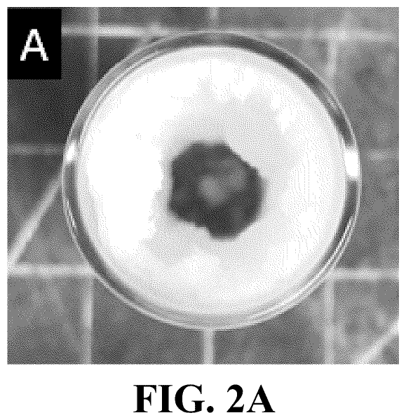

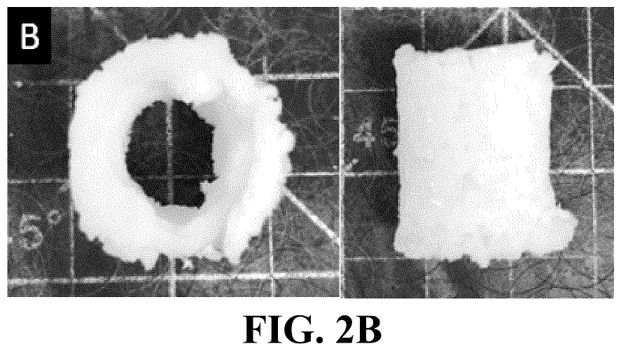

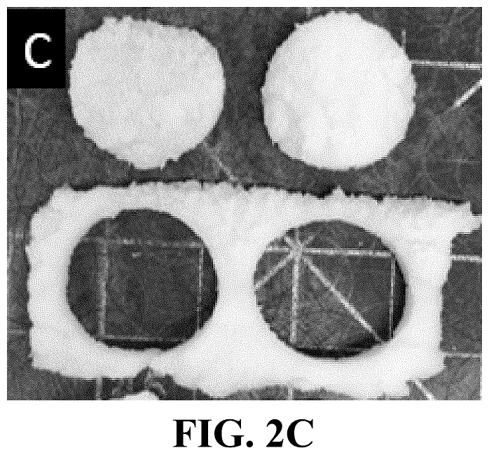

The approach of stepwise polymerization of rings in a rotating glass or polypropylene vials as described in Example 1 was successfully used to fabricate multiple core-annular structures with at least one porous ring. FIG. 2A shows a top view of the single layer polymerized pHEMA hydrogel made with 80% water in the precursor in the glass vial. FIG. 2B shows the top view (left) and side view (right) of the hydrogel tube removed from the vial. The image in FIG. 2C show the film obtained after cutting the porous annulus along the circular films punched out for measuring water content and protein transport in the radial direction.

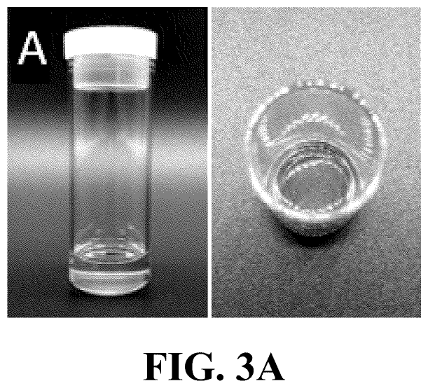

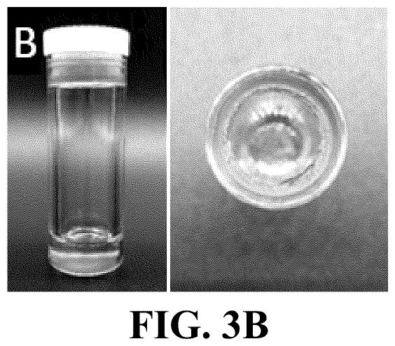

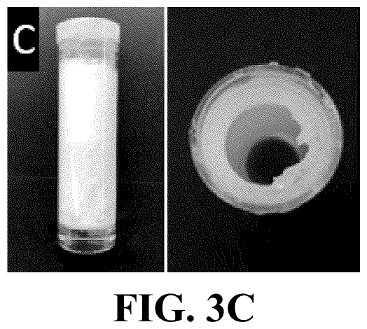

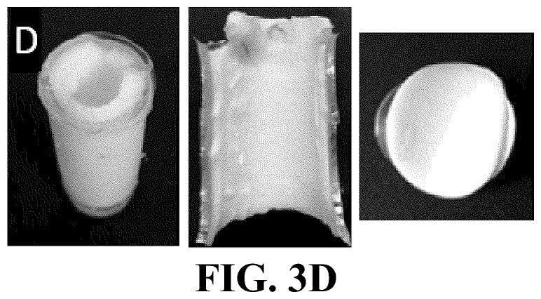

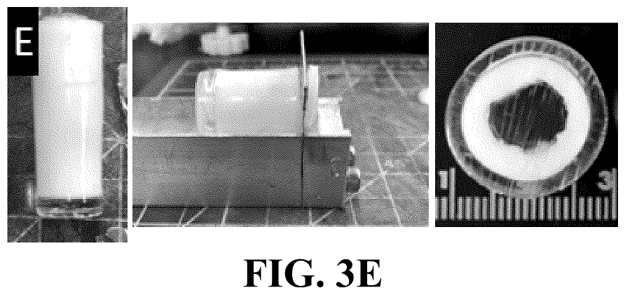

FIG. 3 includes images from various steps of the process developed for obtaining gels that were used for measuring the radial transport with no flux at the bottom surface and axial transport. FIGS. 3A-C include the front view on the left and the top view on the right. The image in 3A shows a clear non-porous pHEMA at the vial bottom. The image in FIG. 3B shows the first step in which a nonporous outermost ring is polymerized, followed by polymerization of the porous ring (FIG. 3C). The image in FIG. 3D shows the two-ring cylindrical tube after removal from the vial (left) and half of the tube after cutting the tube along an axial plane (middle). The image on the right was obtained after punching a circular disc from the half-tube shown in the middle. The disc on the right of FIG. 3D was used to measure the axial transport with the bottom surface blocked to diffusion of the proteins. FIG. 3E (left) shows the image of the rod after the tube shown in FIG. 3D was filled with low water content HEMA monomer formulation and polymerized to yield a transparent core. The rod was cut (middle) into thin discs (right) which were used to measure the axial transport. FIG. 3A shows that a thin layer of non-porous HEMA can be polymerized at the bottom of the vial prior to polymerizing the concentric cylindrical rings. In a few cases, the disc shown in FIG. 3E are cut such that the top surface of the disc included the concentric rings while the bottom surface of the disc was cut through the non-porous layer at the bottom, yielding discs where the diffusion of the biologics occurred only from the top. Thus, we obtained discs for measuring radial transport with protein diffusion from both top and bottom (a single pHEMA layer) and protein diffusion only from the top (FIG. 3D). We also obtained discs for measuring axial transport from both top and bottom (FIG. 3E) and only from the top (diss cut from the bottom of the rod).

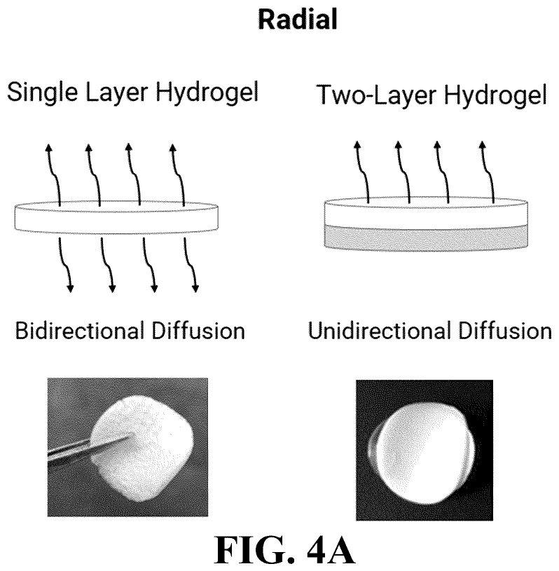

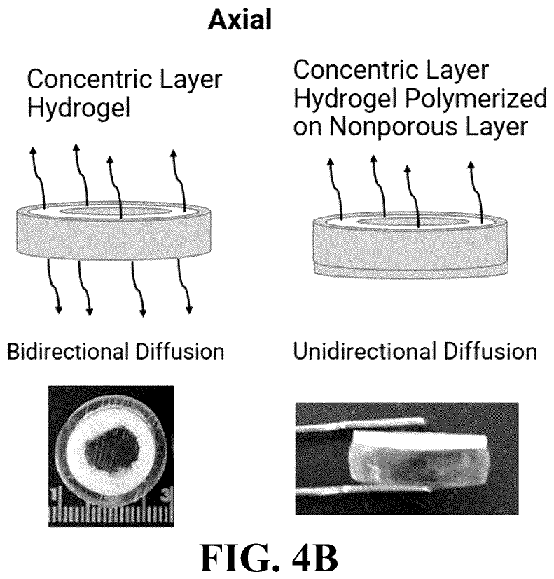

These different disc samples are imaged in FIG. 4 along with schematics illustrating the direction of transport. FIG. 4A shows radial diffusion with a single- and two-layer hydrogel. FIG. 4B shows axial diffusion with a concentric layer hydrogel and a concentric layer hydrogel polymerized on a nonporous layer. The samples with a nonporous backing (unidirectional diffusion) could be useful in applications requiring preferential release in only one direction. For example, a contact lens with unidirectional diffusion could target cornea or conjunctiva.

A single layer tube of the porous gels with thickness of approximately 1.5-1.6 mm was punched to a diameter of 19.05 mm to obtain discs that were utilized to measure radial transport. The concentric layer rods were cut into discs of 2.5 mm length and 5 mm length to measure the axial transport. The gels were loaded with the model proteins and then soaked in fresh buffer to measure the release profiles.

The two-layered gels included a non-porous layer to prevent protein diffusion.

### Example 3: Transport in Layered Hydrogels

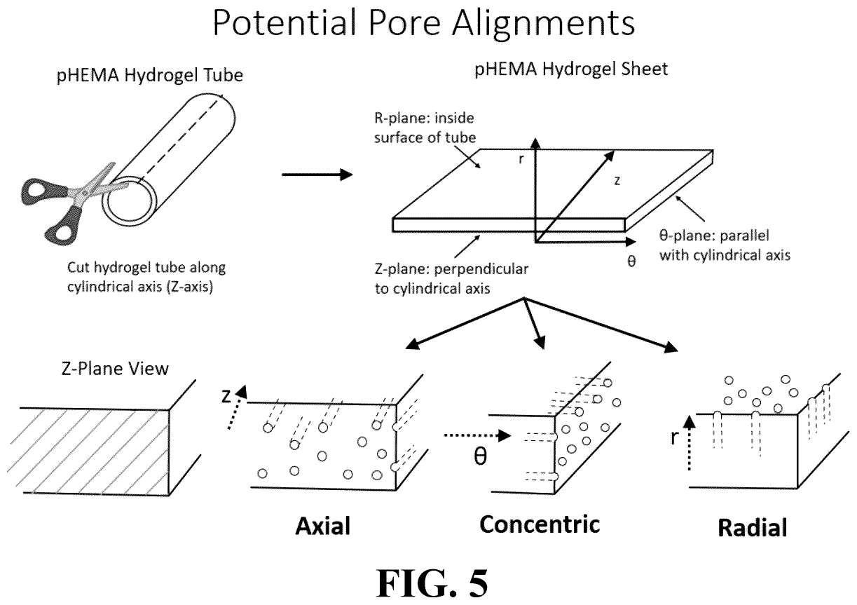

The samples from Example 2 were then used in protein transportation experiments. FIG. 5 shows the different directions in which pores may be oriented.

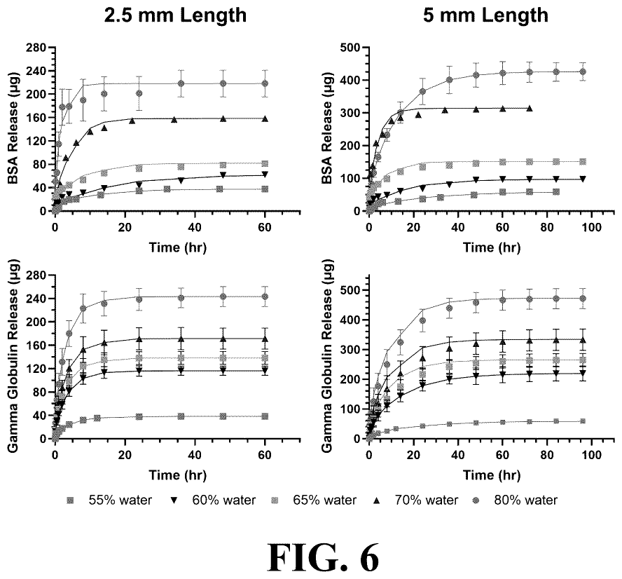

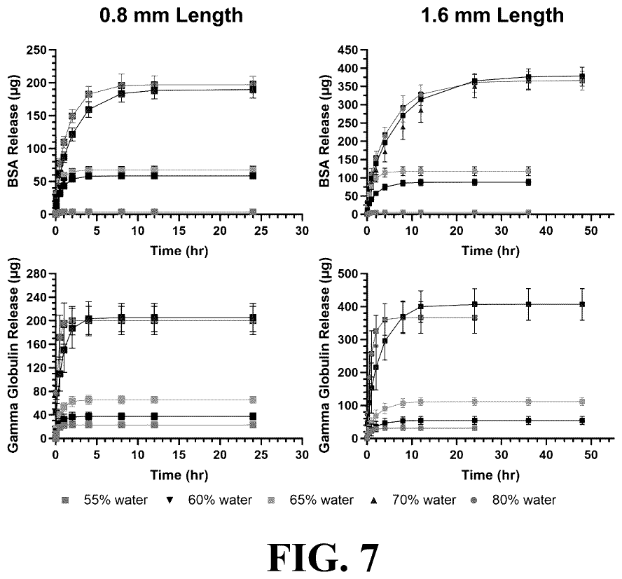

The BSA and gamma globulin release profiles are shown in FIGS. 6 and 7. The dynamic concentration in the release medium was fitted to the sink model to determine diffusivities of the two model proteins in the porous hydrogels. Additionally, the total mass of the protein released from the gels was utilized to determine the concentration of the proteins in the pores, which was divided by the protein concentration in the loading solution to determine the partition coefficient. The D and K values from both radial and axial samples are listed in Table 1 as a function of the water fraction in the formulation. The release studies were conducted with gels of two different thicknesses to ensure that the transport was diffusion controlled.

The fits between the diffusion control sink model (solid lines in FIG. 6 and FIG. 7) and the experimental data are good in all cases which, without wishing to be bound by any theory, is hypothesized that the release is controlled by diffusion of protein through the pores. The mass of the protein released increases with an increase in the water fraction in the formulation due to an increase in the volume of the pores in the polymerized gels. The diffusivities of both gamma globulin and BSA are relatively independent of the direction and the water fraction in the formulation. However, the partition coefficient is higher for the gels designed to measure the radial transport, and additionally the partition coefficient increases with an increase in the water fraction in the formulation.

As shown in FIG. 6, BSA and γ-globulin were released from multi-concentric layer samples, with a length of either 2.5 mm or 5 mm, to measure axial transport of the hydrogel formulations. Model fits are denoted as solid lines and the error bars are standard deviations (n=3).

As shown in FIG. 7, BSA and gamma globulin were released from single layer pHEMA samples, with a length of either 0.8 mm or 1.6 mm, to measure radial transport of the hydrogel formulations. Solid lines denote the model fits and the error bars are standard deviations (n=3).

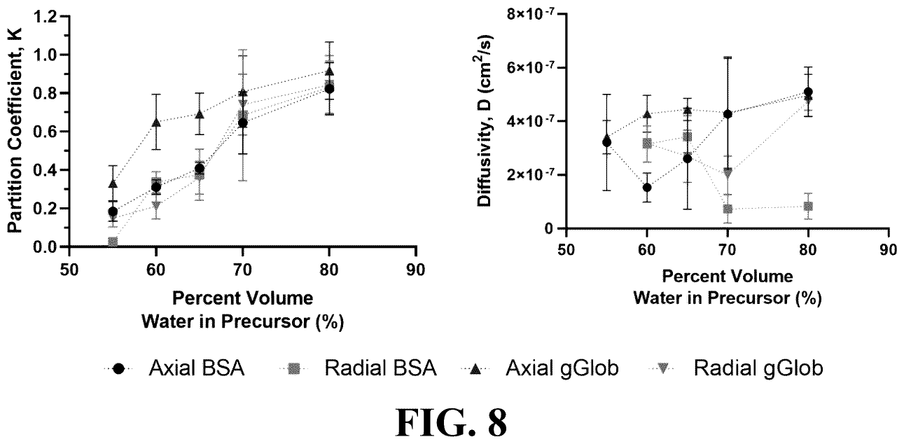

The partition coefficient, K, and diffusivity, D, can be calculated as described in Sparks, Z. et al., “Polymerization of hydroxyethyl methacrylate (HEMA) under rotation to form core-annular hydrogels,” Journal of Colloid and Interface Science (2025). The results for K and D for these pHEMA hydrogels made from formulation varying in water content from 55 to 80% water are tabulated in Table 1 and shown in FIG. 8.

TABLE 1
Partition coefficients, K, and diffusivities, D, were
obtained for porous hydrogel formulations of water content
ranging from 55%-80%. Both axial and radial pore directions
were tested using BSA and gamma globulin.

| | Hydrogel | Water | | |
|---|---|---|---|---|
| | Pore | Content | Partition | Diffusivity, D |
| Protein | Direction | (%) | Coefficient, K | (×10⁻⁷) (cm²/s) |
| BSA | axial | 55 | 0.184 ± 0.052 | 3.21 ± 1.79 |
| | | 60 | 0.310 ± 0.039 | 1.53 ± 0.54 |
| | | 65 | 0.410 ± 0.031 | 2.6 ± 1.89 |
| | | 70 | 0.465 ± 0.091 | 4.27 ± 2.08 |
| | | 80 | 0.823 ± 0.136 | 3.51 ± 0.92 |
| | radial | 55 | 0.025 ± 0.006 | — |
| | | 60 | 0.336 ± 0.055 | 3.15 ± 0.67 |
| | | 65 | 0.375 ± 0.133 | 3.42 ± 0.78 |
| | | 70 | 0.685 ± 0.341 | 0.73 ± 0.53 |
| | | 80 | 0.831 ± 0.136 | 0.83 ± 0.48 |
| γGlob | axial | 55 | 0.331 ± 0.091 | 3.41 ± 0.62 |
| | | 60 | 0.650 ± 0.144 | 4.28 ± 0.07 |
| | | 65 | 0.693 ± 0.109 | 4.44 ± 0.42 |
| | | 70 | 0.808 ± 0.186 | 4.32 ± 0.22 |
| | | 80 | 0.917 ± 0.149 | 4.96 ± 0.79 |
| | radial | 55 | 0.149 ± 0.046 | — |
| | | 60 | 0.211 ± 0.066 | 3.18 ± 0.02 |
| | | 65 | 0.360 ± 0.087 | 2.69 ± 0.97 |
| | | 70 | 0.740 ± 0.158 | 1.99 ± 0.71 |
| | | 80 | 0.843 ± 0.152 | 4.75 ± 0.34 |

### Example 4: Water Content of Hydrogels

Continuing with the samples from Example 3, in some embodiments, the outermost layer comprised a 2/3 v/v water/HEMA formulation and the next layer comprised a HEMA formulation with water content ranging from 11/9 to 4/1 v/v water/HEMA (Table 2) to form the porous annulus. The third layer and the central core were polymerized with water-free HEMA monomer formulation.

The equilibrium water content of the porous gels could be used to calculate the volume of pores in the hydrogel. Table 2 lists the percentage of water (v/v) in the formulation and the calculated equilibrium water content based on the measured dry and wet weights, which were also used to measure the volume of pores (as described in Sparks, Z. et al., “Polymerization of hydroxyethyl methacrylate (HEMA) under rotation to form core-annular hydrogels,” Journal of Colloid and Interface Science (2025)). The volume of pores was divided by the total water volume to calculate the fraction of water that is in the pores. Additionally, the volume of water in pores was divided by the total gel volume to determine the fractional volume that was occupied by pores.

TABLE 2
Water content used in the precursor formulations and calculated
values of the percent water mass in formulation and theoretical
percent pore water to total water in hydrogel.

| Water/HEMA | Percent water | Theoretical |
|---|---|---|
| in precursor | mass in precursor | % pore water/ |
| (v/v) | (%) | total water |
| 2/3 | 38.3 | 0 |
| 11/9 | 53.3 | 12.1 |
| 3/2 | 58.3 | 22.2 |
| 13/7 | 63.4 | 30.8 |
| 7/3 | 68.5 | 38.1 |
| 4/1 | 78.8 | 50 |

The pore water/total water of these samples is reported in Table 3, which also includes the equilibrium water content (EWC) of the different precursor formulation ratios. The theoretical percent of pore water to total water can be calculated as described in Sparks, Z. et al., “Polymerization of hydroxyethyl methacrylate (HEMA) under rotation to form core-annular hydrogels,” Journal of Colloid and Interface Science, which also includes a description on how to calculate the equilibrium water content.

TABLE 3
Water content used in the precursor formulations
and the water uptake in the porous gels.

| | Water/HEMA in | | |
|---|---|---|---|
| | precursor formulation | | Pore Water/Total |
| | (v/v) | EWC (%) | Water (%) |
| Single Layer | 2/3 | 40.7 ± 1.1 | — |
| | 11/9 | 48.1 ± 2.9 | 27.7 ± 10.8 |
| | 3/2 | 51.1 ± 1.0 | 35.9 ± 2.7 |
| | 13/7 | 64.9 ± 1.2 | 49.4 ± 5.2 |
| | 7/3 | 75.1 ± 3.6 | 77.7 ± 1.0 |
| | 4/1 | 82.5 ± 4.7 | 85.0 ± 4.8 |
| Multi-concentric | 11/9 | 42.8 ± 1.0 | 11.0 ± 3.8 |
| Layers | 3/2 | 47.6 ± 3.9 | 25.0 ± 6.6 |
| | 13/7 | 58.9 ± 1.0 | 43.3 ± 5.9 |
| | 7/3 | 49.6 ± 8.2 | 24.1 ± 2.7 |
| | 4/1 | 50.8 ± 1.9 | 35.3 ± 4.9 |

The fully polymerized rod was removed from the glass vial and submerged in excess water for more than 5 days to hydrate the rod. After equilibrium hydration, the hydrogel rod was cut into thin discs ranging from 2-5 mm in thickness and soaked in DI water to extract the unpolymerized monomer, crosslinker and initiator. The DI water was replaced multiple times over 4 or more days to achieve complete extraction of the unreacted components.

The fully hydrated hydrogels were weighed and then allowed to dry under room conditions. The mass of the hydrogel was recorded as a function of time until the weight reached the constant dry weight which was used to calculate the equilibrium water content.

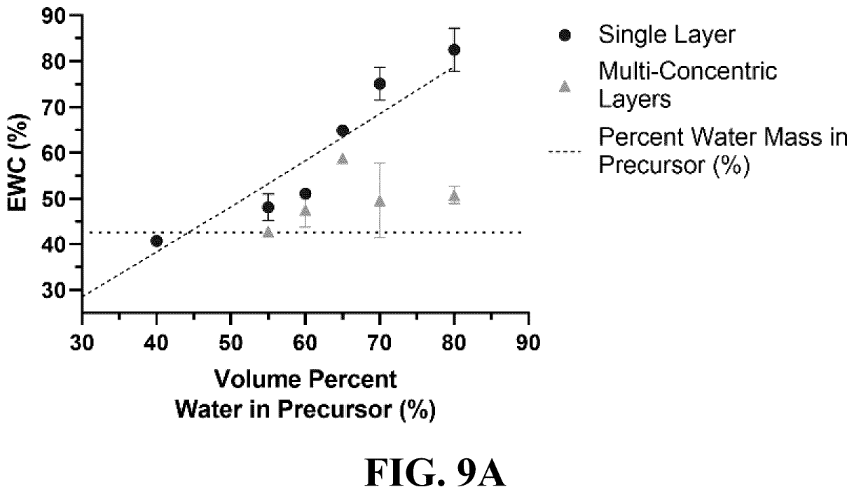

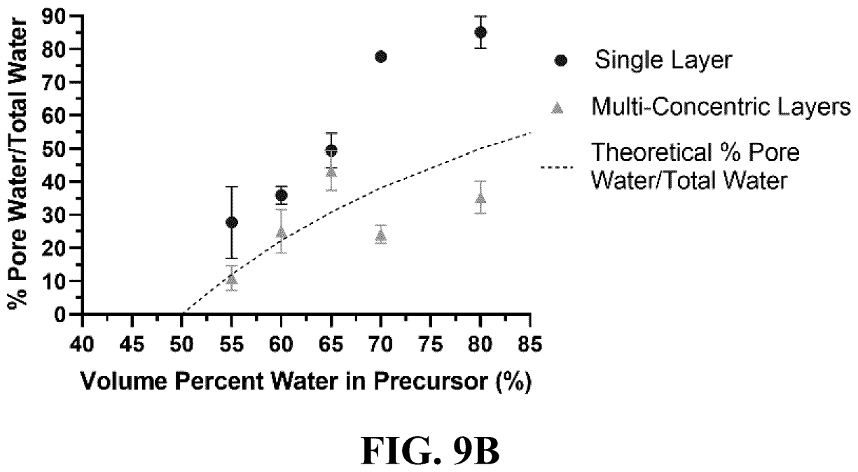

The EWC values of the single layer porous gels in FIG. 9A are close to the percent water mass in precursor line, but there are some deviations which suggests that the composition of the polymerized formulation may differ slightly from the initial formulation possibly due to effects of the rotation. The HEMA formulations are expected to become porous only above water fraction of 60%, but the data in FIG. 9B shows that the gels have finite pore volumes for 55% water in the formulation. This also is likely because of centrifugal forces which may lead to non-homogeneous compositions due to differences in densities of the polymerizing microgels and the unpolymerized formulation. The layer hydrogels which include one porous and one nonporous ring show larger deviations which is also expected because the water content of the non-porous layer remains fixed even though the water content of the porous layer increases with the increase in water content of the formulation.

The rate of water loss from the porous gels is expected to be faster due to the presence of the large pores. The rate of water loss can thus be used to indirectly assess the presence and connectivity of the pores.

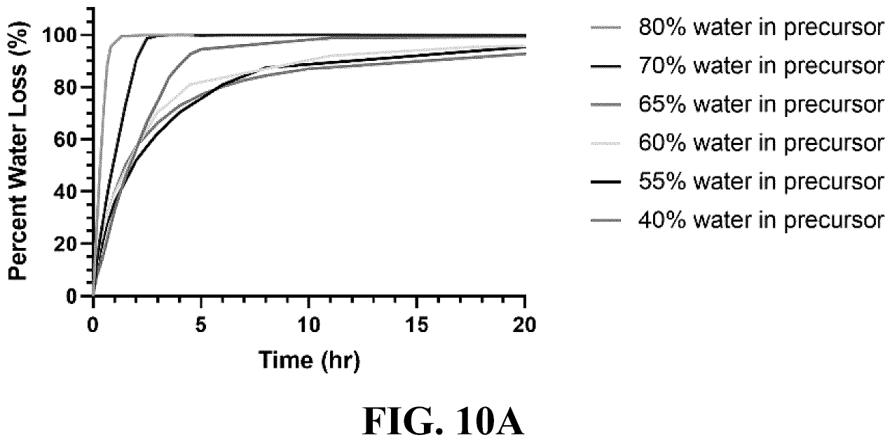

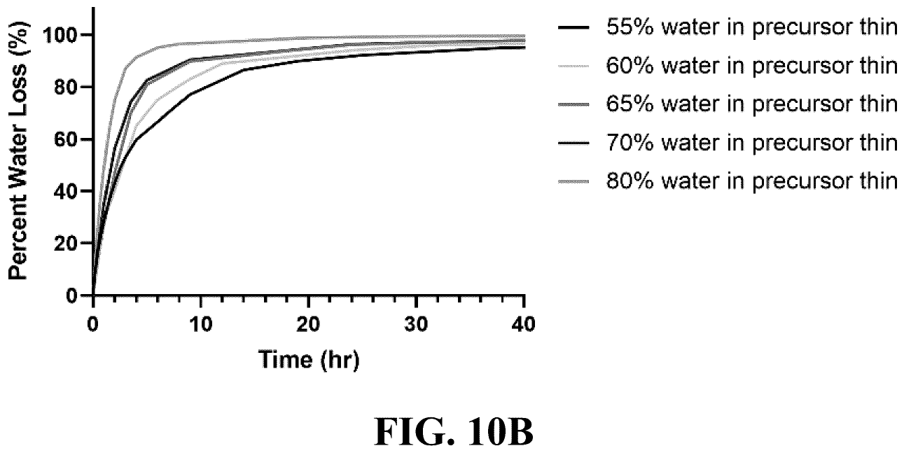

The data in FIGS. 10A and 10B shows that the time required for about 90% loss of water due to drying is much shorter for the porous gels. The drying rate becomes independent of the water content in the formulations for 55% or less. The 60 and 65% gels show a biphasic drying dynamic with a rapid drying at short times due to water loss from the pores followed by a slower water loss from the nonporous regions. Based on the water content data, we expect the gels with 55% water in the formulation to contain pores but the drying data overlaps with the non-porous gels likely because the pores and not connected and thus even the water in pores much diffuse through the nonporous regions. The trends are similar for the two-layer gels in FIG. 5B.

### Example 5: Images of pHEMA Hydrogels with Varying Water Content

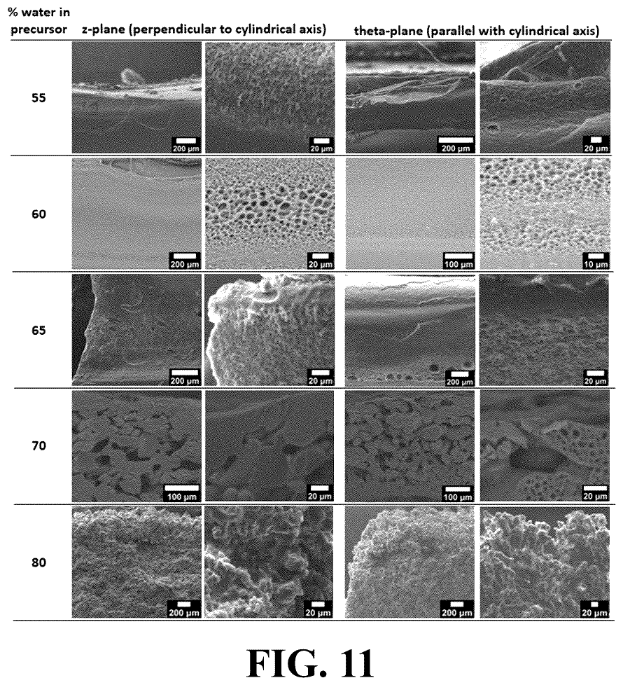

The cross sections of the pHEMA hydrogels made from formulations with water fraction ranging from 55 to 80%, using methods described in previous examples, were imaged using SEM and is shown in FIG. 11.

The porous hydrogels were imaged by Scanning electron microscopy (SEM) (JEOL 7000 FESEM Akishima, Tokyo, Japan) to obtain the detailed microstructure and to observe if there is preferential pore alignment in any direction. The hydrogels were cross-sectioned along the Z-plane and θ-plane for the SEM imaging. The hydrogel samples were placed on an aluminum stub with carbon tape, air dried, and coated with gold for 30 seconds prior to imaging. Samples were imaged using an accelerating voltage of 20 kV.

As shown in FIG. 11, the radial direction extends downward in the images, where hydrogel inner radius is at the top. The left two columns show cross sections of the z-plane, where the z-axis (cylindrical axis) goes into the page. The right two columns show cross sections of the θ-plane which are parallel with the cylindrical axis, where the-axis of goes into the page. The inner right column is a low magnification image, and the outer right column is a high magnification image. The lower magnification image is included to show the entire gel to show radial variations in the samples, while the higher magnification images are included to present the detailed features.

The Z-plane image of the 55% water formulation include small pores a few microns in size but only towards the inner surface of the tubular hydrogel. The region further away, i.e., towards the periphery, away from the center does not contain any pores. The pores are only visible in the higher magnification image due to the small size. This again suggests that the centrifugal forces lead to heterogeneities in composition and pores only form where the local water content exceeds the water content of the saturated HEMA hydrogel. Additionally, the presence of the air-liquid interface at the inner radius may have an impact on the pore formation as well. The θ-plane of the 55% formulation appears to contain less pores which could be due to alignment of the pores in the z-direction. The formulations with 60% or more water appear to be more uniformly porous though the 60% formulation appears to include a more porous band somewhere in between the inner and the outer radius. The 65% water formulation shows that the gel has macropores across the entire radial length and the pores appear more uniform in size particularly in the region closer to the inner. A few much larger pores are present closer to the outer surface. The formulations with 70 and 80% water contain very large, interconnected pores, and addition smaller pores within the HEMA matrix.

### Example 6: Planar Sheet Hydrogel Polymerization

2-hydroxymethacrylate (HEMA), ethylene glycol dimethacrylate (EGDMA), and photoinitiator Darocur Diphenyl (2,4,6-trimethylbenzoyl)-phosphine oxide (TPO) were purchased from Sigma Aldrich. Deionized (DI) water was used in all precursor formulations. Bovine serum albumin (BSA), human gamma globulin, hydroxy propyl methyl cellulose (HPMC), gold chloride, sodium borohydride, and phosphate buffered saline (PBS) were purchased from Sigma Aldrich. Pierce Micro BCA Assay reagents were purchased from Fisher Scientific.

A range of pHEMA gels were prepared with increasing water content to determine the critical value above which the gels turn opaque signifying formation of large pores. pHEMA hydrogels were synthesized by free radical polymerization with photoinitiation. The monomer mixture contained various water contents ranging from 2/3 to 4/1 v/v water/HEMA. Briefly, specific volumes of water (2, 3, 4.5, 7, 12 mL) were added to 3 mL of HEMA monomer. 15 μL of EGDMA crosslinker was added to give 0.5% v/v EGDMA/HEMA. The mixture was purged for 15 minutes with nitrogen to remove oxygen. 6 mg TPO was added for a concentration of 2 mg/ml TPO to HEMA. Hydrogels were polymerized with thicknesses of 250 μm. Molds were irradiated with 305 nm light for 40 min using a MaestroGen transilluminator (LB-16 UltraBright LED Transilluminator, MaestroGen, Hsinchu City, Taiwan).

The planar pHEMA hydrogels were produced with a range of water contents to determine the critical water content required for preparing a porous gel and to access the strength of the material for use in contact lenses. The monomer formulations were prepared with water contents ranging from 40-80% v/v while keeping photoinitiator concentration and crosslinker concentration constant of 2 mg/mL TPO and 0.4% vol. EGDMA, respectively. The gels were prepared by injecting the formulation into a mold prepared by separating two glass plates by a spacer and exposing to UV light in a transilluminator. After polymerization, the hydrogel sheet was removed, and arch punched to obtain circular hydrogel samples about 14 mm in diameter.

### Example 7: Planar Sheet Hydrogel Transmittance

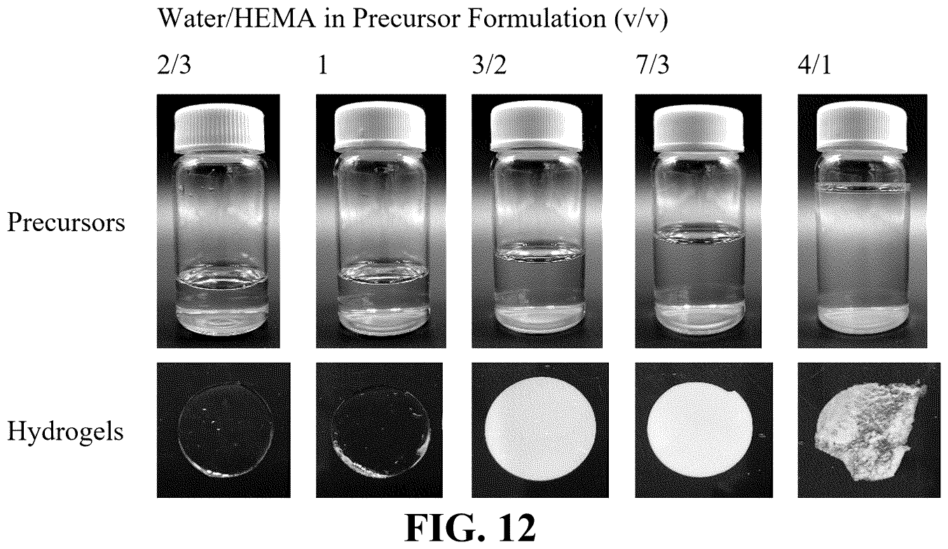

For the samples from Example 6, the transmittance of 250 μm thick hydrated gels was measured using UV-Vis spectrophotometer (Genesys 140/150 Vis/UV-Vis Spectrophotometer, Thermo Fisher) at wavelengths ranging from 300 nm to 900 nm. HEMA hydrogels were prepared with a range of water: HEMA ratio in the mixture. The photoinitiator and crosslinker concentration was kept fixed at 2 mg/mL TPO and 0.4% v/v EGDMA/HEMA respectively. The ratio of water: HEMA was varied from 2/3 to 4/1 v/v. As seen in FIG. 12, the hydrogels made from precursor with 2/3 and 1/1 v/v water/HEMA were clear while hydrogels made from formulations with higher water content are opaque.

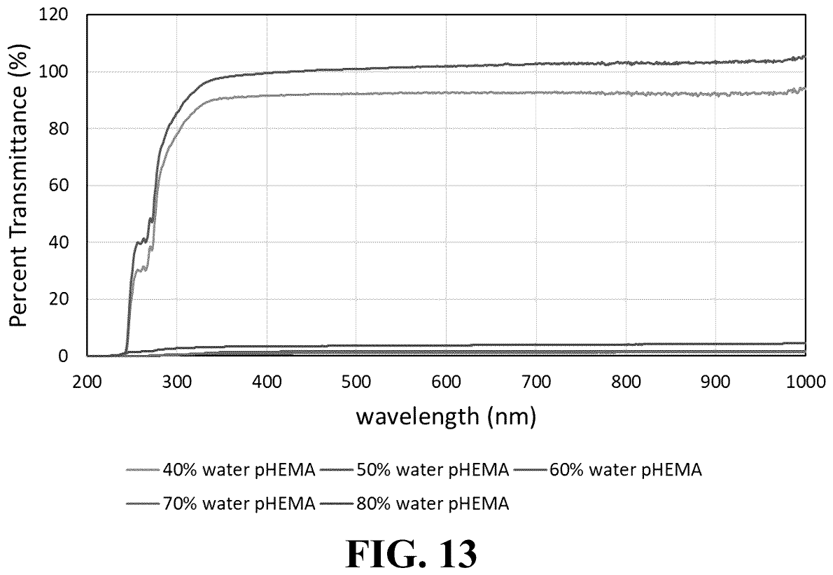

The gels prepared with water/HEMA ratios of 2:3 and 1:1 were transparent and the gels with 3:2 and 7:3 ratios were opaque signifying a porous structure. All gels with water: HEMA ratio of 7:3 and smaller polymerized uniformly, while the formulation with 4/1 water: HEMA ratio polymerized nonuniformly (FIG. 12). The pHEMA hydrogels polymerized with 2:3 and 1:1 v/v water/HEMA ratio are transparent with >90% transmittance in the visible region, while all other gels were white in appearance, and the transmittance for these were below 5% (FIG. 13). All pHEMA hydrogels possessed sufficient physical integrity which suggested that the porous annulus in the contact lenses must be prepared with formulations that contain between 50 and 75% water.

The pHEMA gels were weighted in both dry and hydrated states to determine the water content (Table 4), which can be compared with the water fraction in the formulation to determine if the water in the formulation was uniformly trapped in both nonporous and porous gels.

TABLE 4
Equilibrium water content (EWC) of pHEMA hydrogels prepared
with a range of water fraction in the monomer mixture

| Water Content in Precursor (%) | EWC (%) |
|---|---|
| 50 | 45.9 ± 1.4 |
| 60 | 62.4 ± 0.6 |
| 70 | 71.5 ± 0.7 |
| 80 | 89.4 ± 1.4 |

### Example 8: PHEMA Rod and Lenses with Porous Annulus the Same Materials Used in

Example 6 were used to create pHEMA rod and lenses with porous annulus. The concentric ring hydrogel rods were produced, as shown in FIGS. 1A and 1B, by stepwise polymerizing in a rotating cylindrical glass vial (1.9 cm ID), which was coated with a thin hydrophobic layer to minimize adhesion of the pHEMA layer to the vial. To form the first, i.e., the outermost layer, the vial was partially filled with the monomer solution. The volume of the formulation was calculated based on the desired thickness of the annulus. The glass vial partially filled with the monomer formulation was loaded onto the tube spinning assembly. A DC motor was connected to a power supply which was set to give a spin rate that produced the precursor annulus, which was about 1700 rpm and higher. A light box with mounted CFL UV bulbs was placed over the tube spinning assembly to provide the radiation for initiating the reaction. The duration of polymerization was set at 20 minutes for the outermost layer and increased by 20 minutes each for the successive layers to account for the absorption of light by the already polymerized layers. In most of the designs, three concentric annuli were polymerized, followed by filling the remaining volume with the monomer formulation and polymerizing the rod core while holding it vertically. The outermost layer was prepared with a 40% water formulation to form a transparent outermost ring. The second layer was prepared with a 55% water HEMA formulation to form the porous ring. The third layer as well as the core were prepared with a water-free monomer formulation.

Five concentric lenses were created with different porous layers. Table 5 contains the composition of the HEMA precursor formulations used to polymerize the inner-connected porous layer in this example. All porous formulations were polymerized for one hour.

TABLE 5

| Lens | Porous Layer Formulation |
|---|---|
| 1 | 55% water, 0.5% v/v |
| | EGDMA/HEMA, 1.2% w/v TPO/HEMA |
| 2 | 55% water, 1% v/v |
| | EGDMA/HEMA, 0.6% w/v TPO/HEMA |
| 3 | 55% 2% NaCl, 0.5% v/v |
| | EGDMA/HEMA, 0.6% w/v TPO/HEMA |
| 4 | 55% water, 2% v/v |
| | EGDMA/HEMA, 0.6% w/v TPO/HEMA |
| 5 | 0% water, 0.4% v/v |
| | EGDMA/HEMA, 0.2% w/v TPO/HEMA |

After completion of polymerization, the hydrogel rod was removed from the vial and submerged in water for 7 days or more to allow hydration of the core. After equilibrium hydration, the hydrogel rod was cut into buttons of 6 mm length. The hydrogel discs were again soaked in water to allow for HEMA monomer exchange in which the unpolymerized HEMA monomer in the hydrogels diffused out. The water used for the extraction replaced with fresh DI water multiple times over the 4 or more days, to achieve complete extraction of the unreacted monomer.

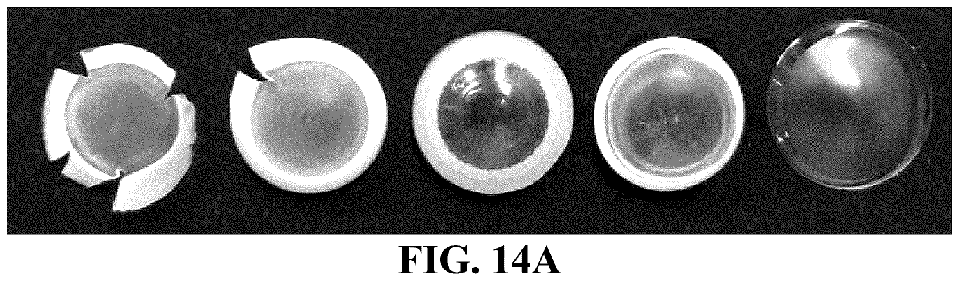

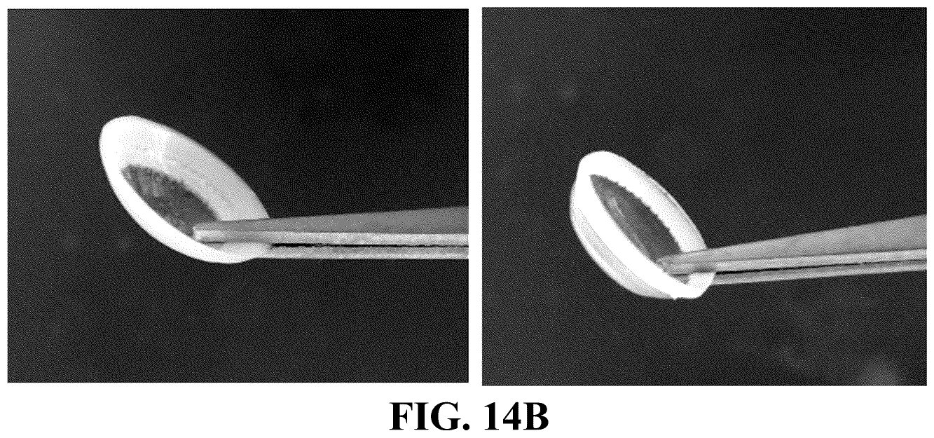

After monomer extraction, the buttons were dried and lathe-cut into contact lenses. The lathe-cut lenses were again soaked in water for extracting any monomer that was not extracted in the previous steps. The lenses, as shown in FIG. 14, were then characterized by measuring water content and transmittance.

The pHEMA rods with clear central zone and a porous annulus were manufactured by conducting stepwise polymerization in a rotating glass mold. After completion of reaction, the rods were taken out from the mold and then cut into shorter cylinders called buttons (FIG. 3E). The buttons were lathe cut to form the contact lenses, about 200 μm thick, which is towards the higher end of the range of thicknesses of commercial contact lenses (FIGS. 14A and 14B). FIG. 14A shows the five lenses tested and FIG. 14B shows lenses 3 and 4 held in forceps for an alternative view.

### Example 9: Characterization of Lathe-Cut Lenses

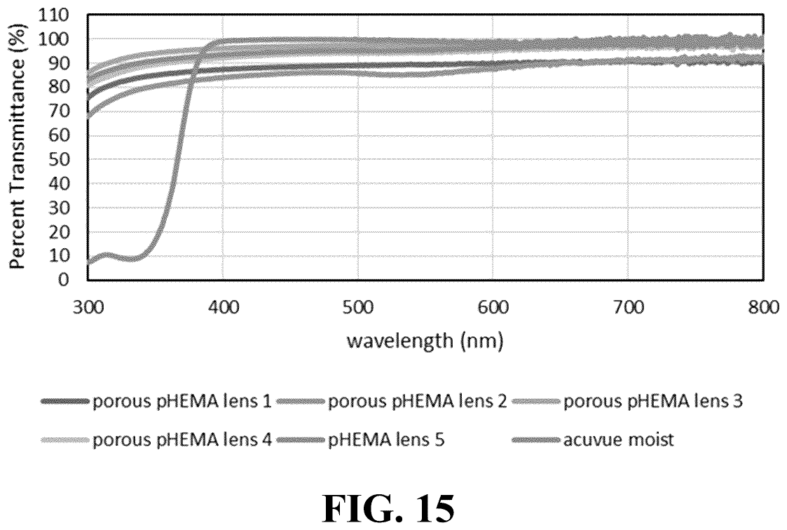

The water content of the lenses from Example 8 were calculated. The dry and wet weight of the lenses were measured to determine the equilibrium water content (EWC) and the volume of pores. The results are in Table 6 and were calculated using methods described in Sparks, Z., et al., “Sustained release of proteins from contact lenses with porous annulus,” Drug Deliv. and Transl. Res. (2025), which is incorporated by reference herein in its entirety. The transmittance of the central portion of the lenses was measured to demonstrate transparency and uses the same methodology as in Example 7, except with 250 μm thick hydrated lathe cut lenses instead of gels. As shown in FIG. 15, the transmittance of lenses 1 and 2 are about 85-90% in the visible range, while the transmittance of lenses 3-5 are greater than 90%, and comparable to the transmittance of Acuvue Moist contact lens.

TABLE 6
Equilibrium water content measurements of lathe cut lenses.

| | wet | dry | water | | pore |
|---|---|---|---|---|---|
| | weight | weight | volume | EWC | volume |
| Lens | (mg) | (mg) | (μL) | (%) | (μL) |
| 1 | 89.2 | 40.8 | 48.4 | 54.3 | 21.2 |
| 2 | 184.5 | 95.7 | 88.8 | 48.1 | 25.0 |
| 3 | 199.2 | 68.6 | 130.6 | 65.6 | 84.9 |
| 4 | 133.5 | 63.8 | 69.7 | 52.2 | 27.2 |
| 5 | 154.6 | 65.9 | 88.7 | 57.4 | 44.8 |

### Example 10: Transport of Model Proteins in Lathe-Cut Lenses

The transport of model proteins was measured and modeled to determine partition coefficient and diffusivity on the lenses from Examples 9.

The contact lenses were loaded with model proteins bovine serum albumin (BSA) and γ-globulin by soaking the lenses in protein solutions at 2 or 10 mg/mL for 14 days at 4° C. Based on the measurements, the 14-day soaking was adequate to achieve equilibrium loading. After 14-days of protein uptake, the lenses were removed from the loading solution and placed in 6.5 mL of PBS to measure the protein release dynamics. The concentration of protein was measured in the release medium at multiple time points. At each time point, 300 μL of buffer were collected, and assayed for protein by using Micro BCA assay. The absorbances of microplate wells were read on a plate reader (Synergy H1 microplate reader, BioTek, Winooski, VT) at 562 nm. The protein release was measured until the concentration in the release medium stopped increasing.

Transport of gold nanoparticles was measured by soaking the lenses in a 2 mg/mL gold nanoparticle dispersion for 14 days. After completion of loading, the lenses were removed from the uptake medium and placed in 6.5 mL of PBS to measure the release dynamics. The concentration of gold nanoparticles was measured in the release medium at multiple time points. At each time point, 300 μL of buffer was collected and assayed for concentration of gold nanoparticles by measuring absorbance at 520 nm using a plate reader (Synergy H1 microplate reader, BioTek, Winooski, VT).

The data from the protein release was utilized to determine the partition coefficient, K, and diffusivity, D, of both proteins. The equations and calculations are described in further detail in Sparks, Z., et al., “Sustained release of proteins from contact lenses with porous annulus,” Drug Deliv. and Transl. Res. (2025). The partition coefficient is the ratio of the concentration of the protein in the hydrogel to the concentration of the protein in solution at equilibrium. The release of the proteins was modeled into 3 mL of aqueous buffer to determine the diffusivity.

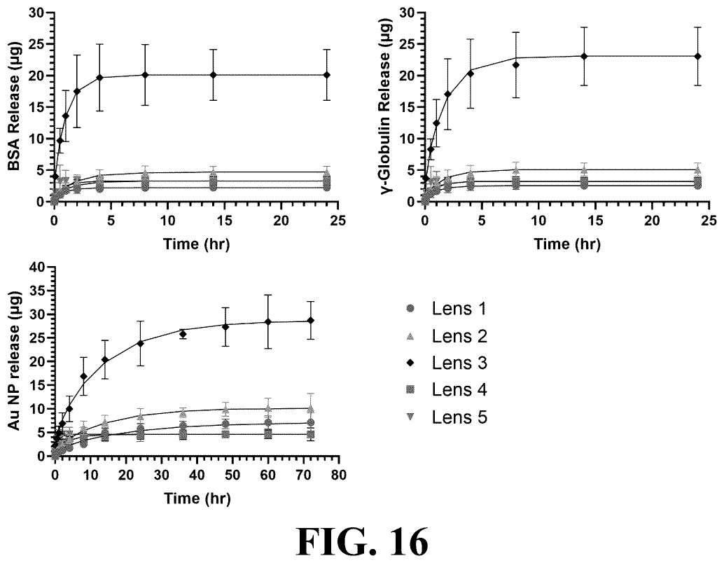

The porous regions were able to load and release model proteins BSA and gamma globulin and gold nanoparticles. The proteins BSA and γ-globulin were loaded into the lenses from 2 mg/mL solutions while Au NP was loaded from a 1 mg/mL uptake solution. The amount of the protein and gold nanoparticle loading varied across the five lenses due to differences in porosity. The release profiles of BSA, γ-globulin, and gold nanoparticles from the five contact lenses are shown in FIG. 16. The release duration of BSA and γ-globulin was about 8 hours while that for the gold nanoparticle was about 40 hours.

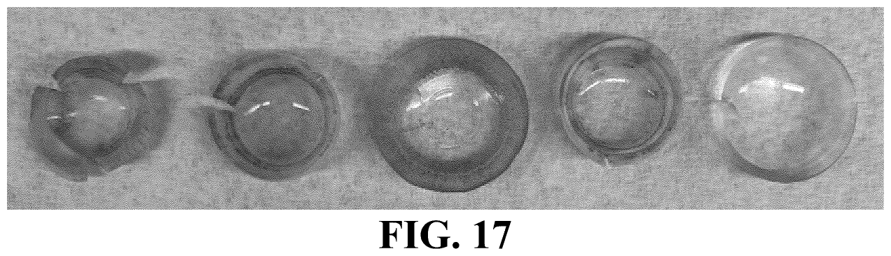

The pHEMA lenses with porous annulus were imaged after soaking in the gold nanoparticle solution (1 mg/ml) for visual evidence of the uptake of the particles and is shown in FIG. 17.

### Example 11: HEMA Hydrogel with Annulus of Magnetic Particles

PLGA microparticles were synthesized using a double emulsion technique to encapsulate fluorescein, a model dye. Various types of PLGA (50:50, 65:35, 75:25, and 85:15) were used and compared for their effect on drug loading and particle size. Amount of stabilizer, Pluronic F68, used in the second water phase of the emulsion was also varied. Results are shown in Table 7.

TABLE 7
Effect of PLGA type and surfactant amount on loading
and size of fluorescein-loaded microparticles

| | | | | Standard |
|---|---|---|---|---|
| | Average | Standard | Average | Deviation |
| | Loading | Deviation | Size | Size |
| | (%) | Loading (%) | (μm) | (μm) |
| 50:50 PLGA 0.25% | 25.8 | 1.8 | 5.5 | 4.7 |
| F68 | | | | |
| 50:50 PLGA 0.5% | 26.1 | 4.6 | 4.9 | 3.7 |
| F68 | | | | |
| 50:50 PLGA 1% | 19.6 | 2.4 | 5.1 | 4.6 |
| F68 | | | | |
| 65:35 PLGA 0.25% | 91.5 | 5.9 | 7.0 | 7.8 |
| F68 | | | | |

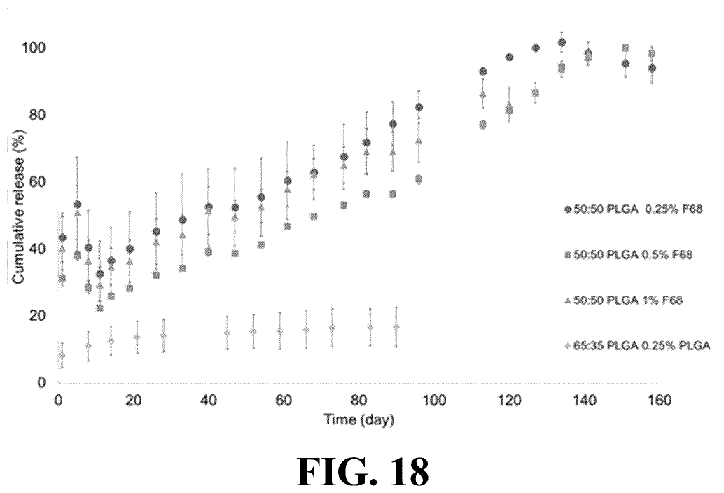

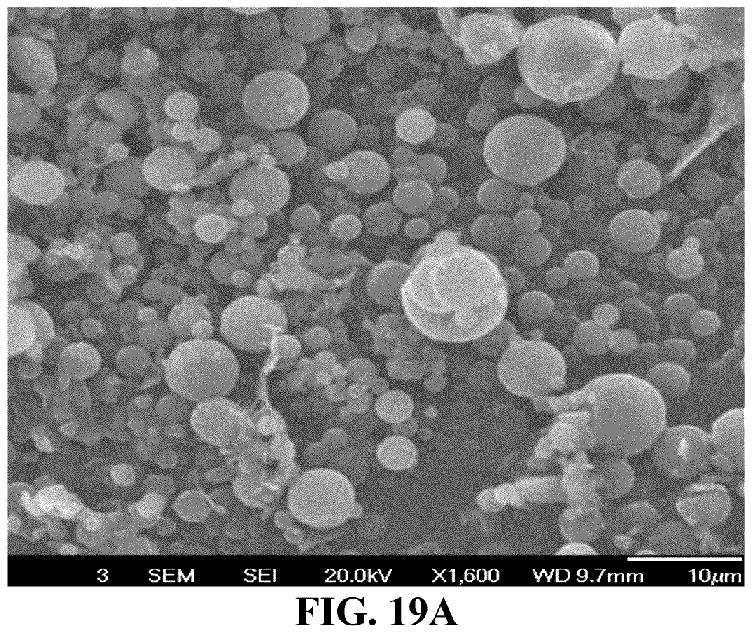

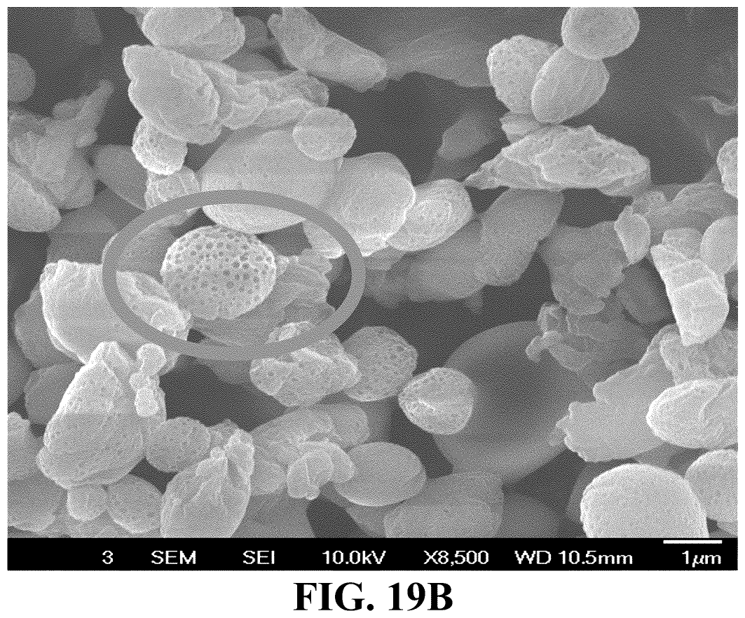

In vitro release of fluorescein is shown from particles up to 120 days as in FIG. 18, which has a mean±standard deviation (n=3). The amount of stabilizer did not significantly affect release profiles, and the polymer type did. SEM images also showed that pore formation began throughout the PLGA particles after 65 days of release in PBS as shown in FIG. 19A and FIG. 19B.

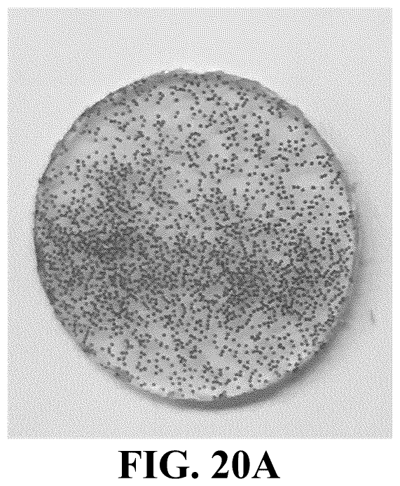

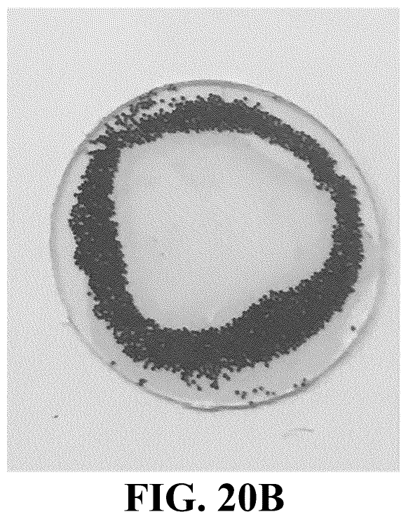

Hydroxyethyl methacrylate (HEMA) lenses were synthesized with loaded magnetic nanoparticles (MNPs) with and without applied magnetic fields. It was demonstrated that with magnetic field application the magnetic microsphere particles can be moved to preferential locations within the lenses as shown in FIG. 20A with a uniform distribution of particles and FIG. 20B, after moving the particles into a ring configuration. When used in some embodiments for novel contact lenses, the ability to control placement of particles can be used to deliver biologics to specific locations on the eye.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1. A method for making an ophthalmic device suitable for drug delivery, comprising:
polymerizing a first monomer formulation in a rotating cylinder to form a first and outermost concentric layer of a contact lens.

2. The method of claim 1, wherein the first monomer formulation comprises hydroxyethylmethacrylate (HEMA).

3. The method of claim 1, further comprising polymerizing a second monomer formulation in a rotating cylinder to form a second concentric layer.

4. The method of claim 3, wherein at least one of the first and second monomer formulations comprises HEMA.

5. The method of claim 3, further comprising polymerizing a third monomer formulation in a rotating cylinder to form a third layer.

6. The method of claim 5, wherein at least one of the first, second, and third monomer formulations comprises hydroxyethylmethacrylate.

7. The method of claim 1, further comprising loading a drug into the contact lens.

8. The method of claim 7, wherein the drug is a biologic.

9. The method of claim 8, wherein the biologic is selected from the group consisting of proteins, micro-RNAs, small molecules, and combinations thereof.

10. The method of claim 7, wherein the biologic has a molecular weight from about 1 kDA to about 180 kDA.

11. A contact lens suitable for ophthalmic delivery of a drug, comprising hydroxyethylmethacrylate and a biologic drug.

12. The contact lens of claim 11, wherein the contact lens comprises three layers.

13. A method for administering a biologic drug to a subject in need thereof, comprising:
applying the contact lens of claim 11 to an eye of the subject, wherein:
the biologic drug is a protein, a micro-RNA, or a combination thereof, the applying step is carried out for at least a duration of release of the drug from the contact lens, and
the duration of release is at least about 2 hours.

14. A contact lens, comprising:
a first layer of a non-porous material; and
a second layer of a porous material,
wherein the first and second layers are concentric.

15. The contact lens of claim 14, further comprising a non-porous, optically clear central portion.

16. The contact lens of claim 15, wherein at least one of the following is true:
(i) a diameter of the central portion is no more than about 8 mm; and
(ii) a visible light transmittance of the central portion is at least about 90%.

17. The contact lens of claim 14, further comprising:
a pharmaceutical drug, loaded within the second layer; and
a third layer of a non-porous material, overlying the first and second layers on a front or outermost surface of the contact lens and configured to promote one-directional diffusion of the pharmaceutical drug into an eye of a wearer of the contact lens.

18. The contact lens of claim 14, further comprising magnetic nanoparticles at one or more predetermined locations within the contact lens.

19. The contact lens of claim 14, having a visible light transmittance of at least about 90%.

20. A method for administering a pharmaceutical drug to a subject in need thereof, comprising:
applying the contact lens of claim 14 to an eye of the subject, wherein the pharmaceutical drug is loaded within the second layer of the contact lens,
wherein the applying step is carried out for at least a duration of release of the drug from the contact lens, and the duration of release is at least about 2 hours.
